(19) United States
(12) Patent Application Publication
(10) Pub. No.: US 2023/0339296 A1
(43) Pub. Date: Oct. 26, 2023

(54) VENTILATION AIR BARRIER SYSTEM FOR ENCLOSED SPACES

(71) Applicant: WAYRAY AG, Zurich (CH)

(72) Inventors: Alexsandr BOBRYSHEV, Zurich (CH); Vitaly PONOMAREV, Zurich (CH); Mikhail SVARICHEVSKII, Zurich (CH)

(21) Appl. No.: 18/250,188

(22) PCT Filed: Oct. 21, 2020
(86) PCT No.: PCT/IB2020/059881

§ 371 Date: Apr. 21, 2023

(51) Int. Cl. B60H 3/06; B60H 1/00; A61L 9/20

(52) U.S. Cl. CPC B60H 3/0608; B60H 1/00021; A61L 9/20; A61L 2209/111; A61L 2209/212; B60H 2003/0675; A61L 2209/12

(57) ABSTRACT

Disclosed embodiments are related to ventilation-based air purification systems, wherein an air purification system generates an air barrier system for an enclosed space. The air purification system provides air barriers between individuals by controlling the air flow between the individuals to prevent passengers' from breathing droplet media/aerosols exhaled by other individuals. Other embodiments may be disclosed and/or claimed.

### FIELD

Embodiments discussed herein are related to medical devices and ventilation systems, and in particular, to air barrier systems for enclosed spaces.

### BACKGROUND

The spreading of a communicable disease at the community, regional, and/or global level can lead to detrimental health effects for individuals who have had or continue to have interactions with infected individuals. Communicable diseases, especially those in which the source of infection can spread through moving air and/or droplet media, such as severe acute respiratory syndrome (SARS)—coronavirus (SARS-CoV or SARS-CoV-1), SARS—coronavirus 2 (SARS-CoV-2), Ebola virus disease (EVD), and influenza have gradually become important issues threatening human health. In particular, novel coronavirus disease 2019 (COVID-19) caused by SARS-CoV-2 has potential for a long-lasting global pandemic, high fatality rates, incapacitated health systems and tremendous economic impact.

Until vaccines become widely available for these communicable diseases, the only available infection prevention approaches are isolation measures, quarantine, physical distancing, decontamination, and hygiene measures. Protective clothing, such as particulate-filtering face piece respirators, cloth face masks, plastic face shields, gloves, etc., may also be used to avoid infection through air or liquid droplet media (i.e., aerosols) between individuals in close contact with one another. However, the isolation effect of using protective clothing along can sometimes be insufficient to prevent cross-infection, depending on the disease and the type of protective clothing used. Additionally, the use of protective clothing is inconvenient and uncomfortable. Another isolation measure involves using physical barriers, such as a plastic separator. However, these physical barriers are difficult to install in some spaces, such as vehicle cabins and the like.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings:

FIG. 1 illustrates a schematic view of an example protective air wall unit (PAWU) implemented in a passenger vehicle, according to various embodiments.

FIG. 2 illustrates a schematic diagram of a gas flow direction of the PAWU of FIG. 1 for passenger isolation, according to various embodiments.

FIG. 3 illustrates a top view of a passenger vehicle implementing the PAWU of FIG. 1, according to various embodiments.

FIG. 4 illustrates a top view of a passenger bus implementing a PAWU, according to various embodiments.

FIG. 5 illustrates an example implementation of a vehicle embedded computer device according to various embodiments.

### DETAILED DESCRIPTION

The following detailed description refers to the accompanying drawings, which are shown, by way of illustration, embodiments that may be practiced. The same reference numbers may be used in different drawings to identify the same or similar elements. Various operations may be described as multiple discrete operations in turn, in a manner that may be helpful in understanding embodiments; however, the order of description should not be construed to imply that these operations are order-dependent. The description may use perspective-based descriptions such as up/down, back/front, top/bottom, and the like. Such descriptions are merely used to facilitate the discussion and are not intended to restrict the application of disclosed embodiments.

In the following description, for purposes of explanation and not limitation, specific details are set forth such as particular structures, architectures, techniques, etc. in order to provide a thorough understanding of various aspects of the embodiments. However, in certain instances, descriptions of well-known elements, devices, components, circuits, methods, etc., are omitted so as not to obscure the description of the embodiments with unnecessary detail. It will be apparent to those skilled in the art having the benefit of the present disclosure that aspects of the embodiments may be practiced in ways that depart from the specific details discussed herein. It should be understood that other embodiments may be utilized and structural or logical changes may be made without departing from the scope of the present disclosure. Therefore, the following detailed description is not to be taken in a limiting sense.

Embodiments discussed herein include ventilation-based air purification systems, and in particular, to air barrier systems for enclosed spaces. The air purification system integrated into a vehicle provides air barriers between individuals by controlling the air flow to prevent individual passengers' from breathing droplet media/aerosols exhaled by other individuals. For example, if a first passenger sneezes, the first passenger's droplets will not flow or pass to a second passenger.

In embodiments, the enclosed space comprises one or more pressure generators (e.g., air knives, air pumps, compressors, vacuum pumps, fan, etc.), ventilation ducting, and one or more air filters (e.g., high-efficiency particulate air/absorbing (HEPA) air filter, electrostatic/plate precipitators, and/or ultraviolet treatment). The air barriers are provided by the pressure generator(s), which generate airflow between individuals in the enclosed space. The airflow of the air barrier flows into the ventilation ducting and passes through the air filter, and then flows out of the ventilation ducting in another part of the enclosed space or outside of the enclosed space. In this way, exhalation from individuals cannot pass to other persons without passing through the filter or other disinfection means, thereby significantly reducing the likelihood of cross-infection. Other embodiments may be described and/or claimed.

The embodiments herein protect individuals in close proximity to one another within enclosed spaces from communicable diseases spread by droplet media and/or aerosols suspended in the air generated from sneezing and breathing in. The embodiments herein allow enclosed spaces, such as vehicle cabins, to avoid using physical barriers/walls between individuals in the enclosed space for preventing communicable diseases from spreading between individuals. Additionally, individuals in the enclosed space do not need to wear personal protective equipment (PPE) when they are inside the enclosed space, even though they may be positioned relatively close to one another. Furthermore, the embodiments herein can be implemented everywhere protective barriers are necessary such as, for example, between passenger seats in a vehicle cabin, in hallways or corridors inside a building, and other places such as those discussed herein.

The enclosed space may be any space that has a limited entry and egress, or otherwise requires individuals to be in close proximity to one another (e.g., within a predetermined distance from one another). As examples, the enclosed spaces may be cabins/cabs of vehicles such as cars, buses, trains, passenger aircraft, aerial trams or cable cars (e.g., “gondola lifts”), and/or the like. Additionally, the enclosed spaces could be building spaces such as relatively small rooms, open offices/workspaces, rooms with multiple cubicles, prison or jail cells, and/or the like. Furthermore, the embodiments herein could also be used for partially enclosed spaces such as bus stop shelters, or non-enclosed spaces such as chairlifts and the like.

### 1. EXAMPLE EMBODIMENTS

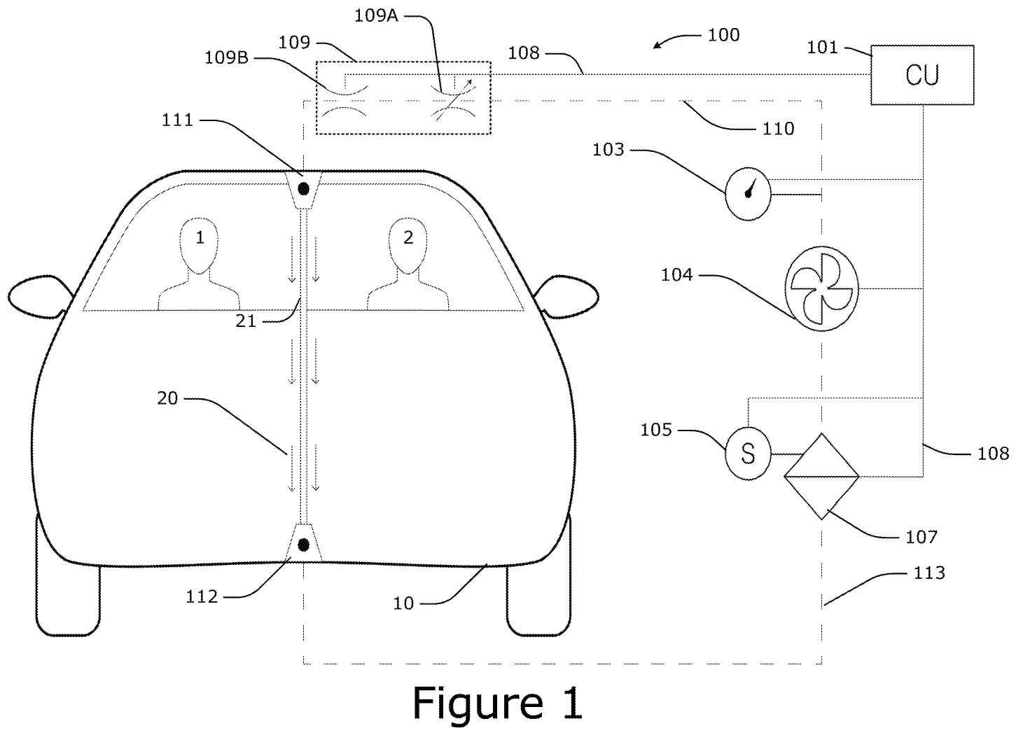

FIG. 1 illustrates a schematic view of an example protective air wall unit (PAWU) 100, according to various embodiments. In this example, the PAWU 100 is implemented in a car 10 with passenger 1 and passenger 2, however, the PAWU 100 may be implemented in other vehicles (e.g., trucks, buses, watercraft, aircraft, etc.) and/or other enclosed spaces (e.g., elevators, office space, etc.) with any number of passengers or users.

In various embodiments, the PAWU 100 comprises a control unit (CU) 101, a gauge/sensor 103, a pressure generator 104, a sensor 105, and a decontamination device 107. In some embodiments, the PAWU 100 further includes a flow regulator 109. In other embodiments, the flow regulator 109 may be disposed in other areas of the car 10. The PAWU 100 is coupled to a ducting system that includes an air duct 110 (also referred to as a “fresh air supply line 110” or the like), a top nozzle 111 (also referred to as an “output nozzle 111”), a bottom nozzle 112 (also referred to as an “input nozzle 112”), and an air duct 113 (also referred to as a “recirculated air supply line 113” or the like). Additionally, a signal line 108 couples the CU 101 to each of the gauge/sensor 103, the pressure generator 104, the sensor 105, the decontamination device 107, and the flow regulator 109.

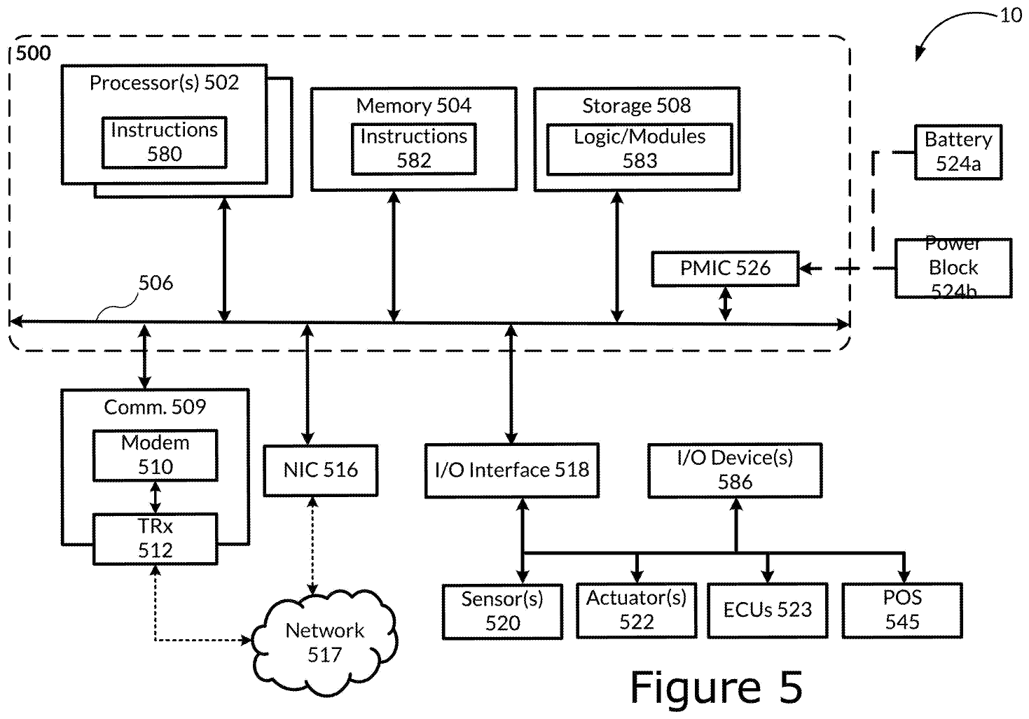

The CU 101 is a device that controls the amount of air, flow strength, flow rate, and/or direction of the airflow 20 throughout the system 100. The CU 101 is operable or configurable to provide varying amounts of current (or varying pulses of current) to the components of the system (e.g., gauge/sensor 103, pressure generator 104, sensor 105, and flow regulator 109, and potentially decontamination device 107 (depending on the type of decontamination device 107 being used)) in order to control the strength and direction of the airflow 20. Different airflow 20 strengths may provide different strengths of the air barrier 21. The control signaling provided by the CU 101 may be achieved using various combinations of current pulses, for example, using phase offset modulation, pulse-width modulation, and/or other like modulation schemes via the signal line 108. The CU 101 may be any suitable computing device (see e.g., system 500 of FIG. 5, an electronic control unit (ECU) 523 of FIG. 5, an actuator 522 of FIG. 5, or the like), such as a microprocessor, microcontroller, or special-purpose processor specifically built and configurable to control the pressure generator 104. The Well-known power/ground connections to connect power source(s) to integrated circuit (IC) chips and other components are not shown within FIGS. 1-5 for simplicity of illustration and discussion, and so as not to obscure the disclosure the illustrated embodiments.

The pressure generator 104 may be any mechanism that generates or otherwise provides an airflow 20 in a predetermined direction, which is indicated by the arrows of airflow 20 in the example of FIG. 1. The airflow 20 flowing between passengers 1 and 2 creates an air-barrier 21. Examples of the pressure generator 104 include air knives, air pumps, air compressors, vacuum pumps, fans, and/or the like and/or combinations thereof. In implementations where air knives are used, the pressure generator 104 may provide a high-intensity, uniform sheet of laminar airflow 20 (sometimes known as a “streamline flow”).

The gauge/sensor 103 (or “pressure gauge 103”) may be a device configured to measure various data/information about the pressure generator 104 such as, for example, pressure levels, power levels, energy consumption, temperature of the pressure generator 104, temperature of the airflow 20, etc. The gauge/sensor 103 may also be configured to deliver system control (e.g., to start/stop the pressure generator 104), along with protection from overheating and the like. In embodiments, the pressure generator 104 processes air from inside and/or outside the vehicle 10 to supply the air for the airflow 20. Once air pressure (or compressed air) or streamline flow reaches a certain pressure point, the pressure generator 104 turns itself off or the CU 101 turns the pressure generator 104 off. The gauge/sensor 103 may comprises one or more electronic sensors and switches that may be used to detect the pressure of the airflow and/or shut the pressure generator 104 off when desired. The gauge/sensor 103 may be any of a variety of measuring instruments or devices configured to monitor the airflow 20 traveling through the system and report measurements of the airflow 20 to the CU 101. Examples of the gauge/sensor 103 include a suitable pressure sensor such as a metal strain gauge, a silicon piezoresistive pressure sensor, a silicon piezoelectric pressure sensor, a gauge pressure sensor, vacuum pressure sensor, capacitive pressure sensor, electromagnetic pressure sensor, thermal conductivity pressure sensor, capacitive pressure sensor, magnetic pressure sensor, optical pressure sensor, and/or the like.

The decontamination device 107 may be any device comprising means to remove contaminants and/or solid particulates from the airflow 20 or other gas such as dust, pollen, mold, droplet media, aerosols, bacteria, virus vectors, and/or other contaminants. The decontamination device 107 may comprise one or more fibrous or porous materials, and in some implementations the decontamination device 107 may include an adsorbent or catalyst such as charcoal or carbon to remove odors and gaseous pollutants such as volatile organic compounds, ozone, and/or the like.

In one example, the decontamination device 107 is a suitable filter such as a HEPA air filter capable of removing at least 99.95% of particles with a size greater than or equal to 0.3 micrometers (μm) as defined by the European Standard EN 1822-1:2009, “High efficiency air filters (EPA, HEPA and ULPA)” (Comite Europeen de Normalisation 2009) and/or American Society of Mechanical Engineers (ASME) AG-1a-2004, “Addenda to ASME AG-1-2003 Code on Nuclear Air and Gas Treatment” (ASME 2004). In another example, the decontamination device 107 is an Ultra-Low Particulate Air (ULPA) air filter capable of removing at least 99.999% of particles with a size greater than or equal to 0.1 μm as defined by EN 1822-1:2009. Additionally or alternatively, the decontamination device 107 may be made of one or more materials such as, for example, paper, polyurethane foam, cotton, stainless steel mesh, fiberglass, semi-HEPA filters, semi-ULPA filters, and/or other like filters.

Additionally or alternatively, the decontamination device 107 may include an electrostatic precipitator (ESP) or plate precipitators (sometimes referred to as “air purifiers”) comprising, for example, a row of vertical wires and a stack of flat metal plates oriented vertically that are spaced apart by about 1 centimeter (cm) to 18 cm apart. In these embodiments, the air stream 20 flows horizontally through the spaces between the wires, and then passes through the stack of plates. A relatively high voltage is applied to produce an electric corona discharge that ionizes the air around the electrodes, which then ionizes the particles in the airflow 20, and the electrostatic force diverts the ionized particles towards the grounded plates. Particles that build up on the collection plates are removed from the airflow 20.

In other embodiments, the decontamination device 107 may additionally or alternatively include an ultraviolet (UV) disinfection system providing UV germicidal irradiation (UVGI). The UVGI system may comprise one or more UV light-emitting diodes (LEDs) configured to emit light with a wavelength in the ultraviolet light range of approximately 10 nanometers (nm) to 400 nm. In some implementations, the UV LEDs may emit short wave UV light in the 100 nm to 280 nm range (also referred to as “ultraviolet C” or “UVC”). In these embodiments, the UV LEDs emit UV light onto the airflow 20 to create an inhospitable environment for microorganisms such as bacteria, viruses, molds, and/or other pathogens.

The sensor 105 is used to monitor the quality and/or operation of the decontamination device 107, and reports relevant sensor data to the CU 101. In embodiments where the decontamination device 107 is a filter, the sensor 105 is a pressure sensor that measures pressure drop across the filter 107 for determining whether the filter 107 is filtering air within its predefined range of effective utilization. In these embodiments, the sensor 105 may be a suitable pressure sensor such as a metal strain gauge, a silicon piezoresistive pressure sensor, a silicon piezoelectric pressure sensor, a gauge pressure sensor, vacuum pressure sensor, capacitive pressure sensor, electromagnetic pressure sensor, thermal conductivity pressure sensor, capacitive pressure sensor, magnetic pressure sensor, optical pressure sensor, and/or the like. Additionally or alternatively, the sensor 105 may be a microwave cavity sensor such as those discussed in Mason et al., “HEPA Filter Material Load Detection Using a Microwave Cavity Sensor”, Int'l J. on Smart Sensing and Intelligent Sys., vol. 3, no. 3 (September 2010).

Additionally or alternatively, the sensor 105 may include a particle sensor for environmental gas detection including, for example, Automotive Air Quality Sensors (AQS) sensors, Interior/Indoor Air Quality (IAQ) sensors, pellistor and/or microelectromechanical systems (MEMS) or nanoelectromechanical systems (NEMS) pellistor sensors (mPell), IR Source and Module (MINIS), MEMS or NEMS thermal conductivity sensors (e.g., thermal conductivity detector (TCD), and/or the like.

Additionally or alternatively, the sensor 105 may include one or more electrochemical nanosensors configured to detect viruses or other infectious agents. In these embodiments, the nanosensor(s) comprise a transducer, a bioreceptor, and a detector wherein a target molecule connects with the bioreceptor and a biological detecting component of the bioreceptor identifies a biological molecule through a reaction, and the transducer converts changes to a signal quantified by the detector. Details of such nanosensors 105 are discussed in detail in Farmani et al., “Nanosensors for Smart Cities”, Elsevier Science (13 Feb. 2020) and Saylan et al., “Virus Detection Using Nanosensors”, Nanosensors for Smart Cities, Elsevier Science, pgs. 501-511 (14 Feb. 2020), each of which are hereby incorporated by reference in their entireties.

Additionally or alternatively, the sensor 105 may be one or more Plasmonic Photothermal Biosensors as discussed in Qiu et al., “Dual-Functional Plasmonic Photothermal Biosensors for Highly Accurate Severe Acute Respiratory Syndrome Coronavirus 2 Detection,” ACS Nano 2020, 14, 5, pgs. 5268-5277 (13 Apr. 2020), which is hereby incorporated by reference in its entirety.

In either of the aforementioned embodiments, the sensor 105 is configured to detect the amount or levels of pressure, particle, and/or infectious agent loads, and provide a digital output to CU 101. The CU 101 may use this information (in addition to the airflow 20 measurements provided by the gauge/sensor 103) to control the airflow 20 via the pressure generator 104.

The flow regulator 109 (or “airflow regulator 109”, “airflow control dampers 109”, “pressure regulator 109”, and/or the like) is a modulating device that automatically regulates fluid or gas to a desired value, and/or provides pressure setting and control. The flow regulator 109 may use feedback of the regulated pressure as input to the control mechanism (e.g., restricting element 109A), which is used to maintain the set regulated pressure. In some embodiments, the flow regulator 109 may be a pressure reducing regulator that reduces the input pressure of a fluid or gas to a desired value at its output (e.g., the output of the pressure generator 104), which is installed upstream of pressure sensitive element (e.g., the nozzle 111).

In this embodiment, the flow regulator 109 includes a restricting element 109A and a measuring element 109B. The restricting element 109A may be a mechanical device that that can provide a variable restriction to the airflow 20, such as a suitable value (e.g., globe valve, butterfly valve, etc.). A loading element may also be included (not shown by FIG. 1), which is configured to apply the needed force to the restricting element 109A. This loading can be provided by a weight, a spring, a piston actuator, diaphragm actuator, electrical actuator, or an actuator in combination with a spring. In some embodiments, the restricting element 109A may be one or more modulating devices. In one example implementation, the restricting element 109A is a device that regulates the size of an opening or orifice through which the airflow 20 flows (e.g., a “modulating orifice”). In this example, the restricting element 109A may be a mechanical device including an aerofoil or aero-wing damper that lifts in response to increasing static pressure or lowers in response to decreasing pressure. In another example implementation, the restricting element 109A may be an electro-mechanical device including one or more actuators that changes the size of the opening or orifice based on commands from the CU 101. In some embodiments, the restricting element 109A may be a flow control value that such as a one way flow control valve that regulates air flow in one direction comprising, for example, a combination throttle valve connected to check valve.

The measuring element 109B (also referred to as a “flow meter 109B”) indicates the flows of the airflow 20 flowing through the flow regulator 109. In some embodiments, the measuring element 109B functions to determine a predetermined or predefined flow level. In some implementations, the measuring element 109B may include a barrier or diaphragm and a certain amount of the airflow 20 may be needed for the measuring element 109B to operate. The flow meter 109B may be a mechanical and/or electronic device that measures the airflow 20, or how much air is flowing through the ducting 110 and/or 113. In one example, the flow meter 109B may be an anemometer configured to measure a speed of the airflow 20. The anemometer may be a straight probe, rotating vane, and/or hot-wire anemometer and may include ultrasound or resistive wires to measure the energy transfer between the measurement device and the passing particles. In one example, the flow meter 109B may be the same or similar as a mass (air) flow sensor (MAF) reconfigured to determine the mass flow rate of air exiting the pressure generator 104 and/or the entering the ducting 110 and/or 113 rather than a fuel-injected internal combustion engine. Other types of flow meters 109B may be used in other embodiments.

The flow regulator 109 may be an integral device where the restricting element 109A and measuring element 109B are contained within a single body, or the restricting element 109A and measuring element 109B may be communicatively coupled with each other but provided/disposed separate from one another (i.e., in respective housings or bodies).

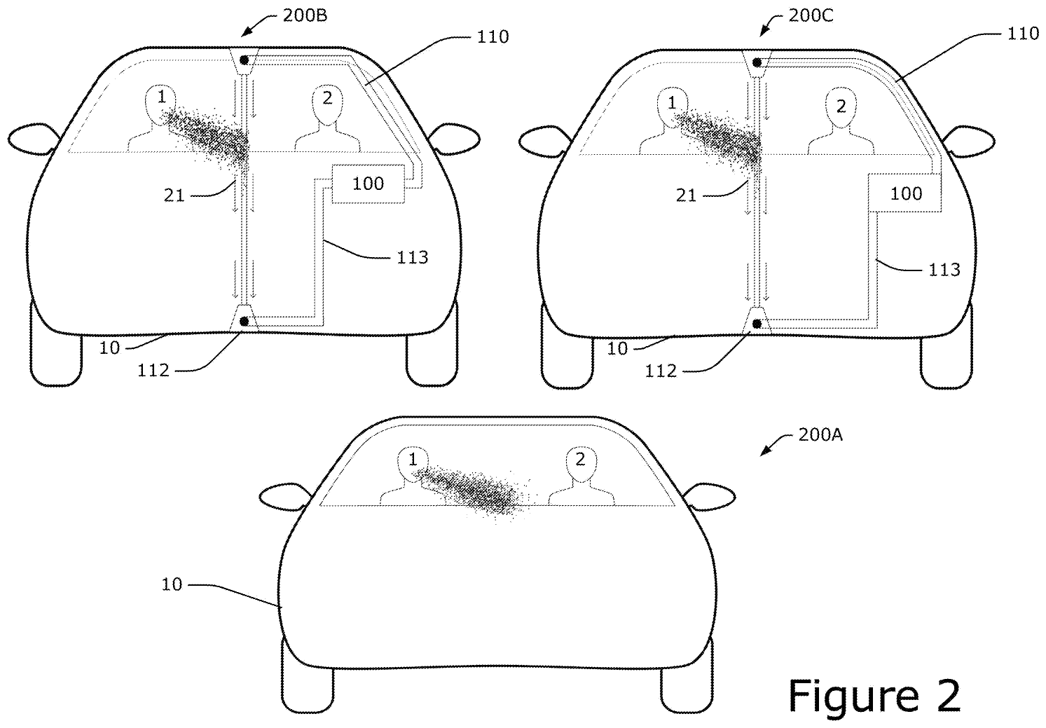

In operation, the CU 101 controls the pressure generator 104 to generate the airflow 20, which travels through supply line 110 out of top (output) nozzle 111 between passengers 1 and 2 creating an air-barrier 21. The airflow 20 then passes through the bottom (input) nozzle 112 and the air duct 113 to the filter 107, and is recirculated through the air duct 110 after particulates and/or infection agents are captured by the filter 107. While the airflow 20 is being generated, the sensor 105, the flow meter 109B, and/or the gauge/sensor 103 provide respective sensor data to the CU 101. The CU 101 increases or decreases the flow amount, flow strength, flow rate, and/or flow direction of the airflow 20 based in part on the data obtained from the sensor 105, the flow meter 109B, and/or the gauge/sensor 103. Additionally or alternatively, the flow regulator 109 regulates the airflow 20 according to its physical/mechanical and/or operational parameters. In this way, a physical barrier is not needed to protect the passengers 1 and 2 from infectious agents potentially harbored by either of the passengers 1 and 2. The air barrier 21 provided between passengers 1 and 2 protects the passengers 1 and 2 in the vehicle 10 from particles or from viruses, as is shown by FIG. 2.

FIG. 2 illustrates a schematic diagram of a gas flow direction of the PAWU 100 for various scenarios 200A, 200B, and 200C, according to various embodiments. In scenario 200A, the vehicle 10 does not include a PAWU 100. When passenger 1 creates an aerosol (e.g., when speaking, sneezing, coughing, etc.), the droplet media may travel towards passenger 2. If the aerosol is carrying a communicable disease, the droplet media could potentially infect passenger 2.

In scenarios 200B and 200C, the vehicle 10 does include a PAWU 100. When passenger 1 creates an aerosol, the air barrier 21 blocks the transmission of the droplet media to passenger 2. The air barrier 21 also carries the droplet media through the bottom (input) nozzle 112 and then through the filter 107 (not shown by FIG. 2).

Scenarios 200B and 200C provide the same air barrier 21 and provide the same isolation among vehicle passengers 1 and 2. However, scenarios 200B and 200C show different PAWU 100 configurations. In particular, scenario 200B shows the duct 113 being attached to a first side of the PAWU 100 and the duct 110 being attached to a second side of the PAWU 100. Scenario 200C shows the duct 113 being attached to a bottom or underside of the PAWU 100 and the duct 110 being attached to a top or upper side of the PAWU 100. Other PAWU 100 configurations are possible, for example, the duct 113 may be attached to a bottom or underside of the PAWU 100 and the duct 110 may be attached to a side of the PAWU 100, or the duct 113 may be attached to a side of the PAWU 100 and the duct 110 may be attached to a top or upper side of the PAWU 100.

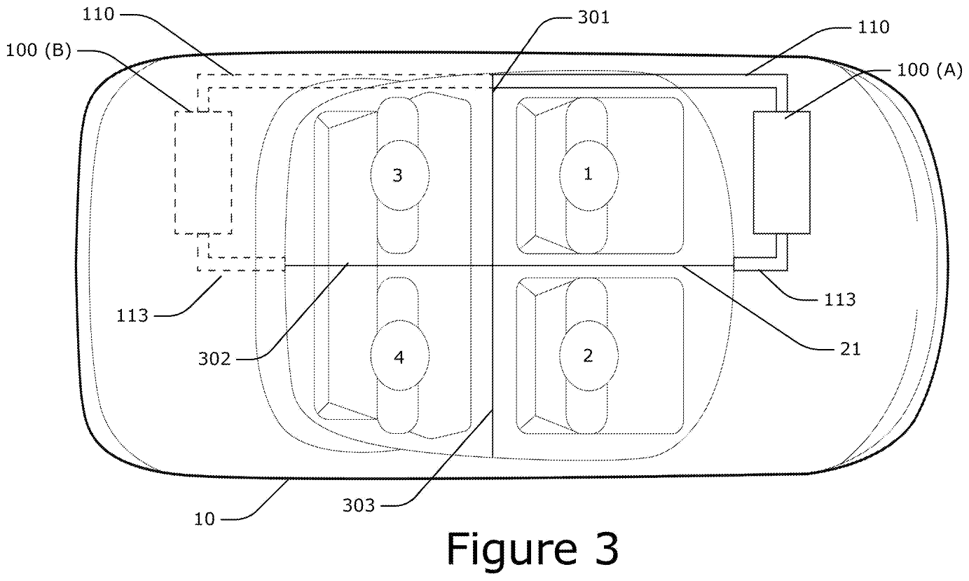

FIG. 3 illustrates a top view of a passenger vehicle implementing the PAWU 100 of FIG. 1, according to various embodiments. FIG. 3 shows potential placement or installation positions of the PAWU 100 within a vehicle 10. In one embodiment, the PAWU 100 may be disposed or installed in a front or forward position of the vehicle 10 (e.g., indicated as PAWU 100 (A) in FIG. 3). In another embodiment, the PAWU 100 may be disposed in a back or rear position of the vehicle 10 (e.g., indicated as PAWU 100 (B) in FIG. 3).

In each embodiment, the ducts 110 and 113 are shown as being attached to the sides of the PAWU 100, however, the ducts 110 and 113 may be attached to the top and/or bottom of the PAWU 100 as shown by FIG. 2.

Furthermore, in the embodiment shown by FIG. 3, additional ducting systems (e.g. including additional ducts 110 and 113 and/or nozzles 111 and 112) to provide another air barrier 301 between passenger 1 and passenger 3, provide another air barrier 302 between passenger 3 and passenger 4, and/or provide another air barrier 303 between passenger 4 and passenger 2.

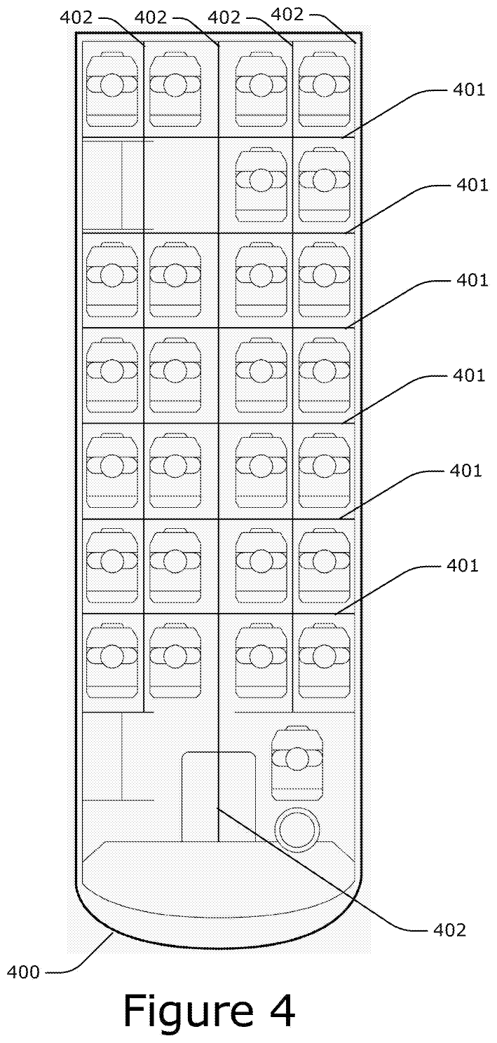

FIG. 4 illustrates a top view of a passenger bus 400 implementing one or more PAWUs, according to various embodiments. The one or more PAWUs implemented in passenger bus 400 may be the same or similar as PAWU 100. In this embodiment, the one or more PAWUs with various ducting systems (e.g., each including nozzles 111 and 112 and/or ducts 110 and 113) to provide multiple air barriers 401 and 402 between the various passengers depicted by FIG. 4. In FIG. 4, each of the air barriers 401 are provided in a lateral direction (e.g., side to side), and each of the air barriers 402 are provided in a longitudinal direction.

Each of the elements/components shown and described herein may be manufactured or formed using any suitable fabrication means, such as those discussed herein. Additionally, each of the elements/components shown and described herein may be coupled to other elements/components and/or coupled to a portion/section of the vehicle by way of any suitable fastening means, such as those discussed herein. Furthermore, the geometry (shape), position, and/or orientation of the elements/components shown and described herein may be different from the depicted shapes, positions, and/or orientations in the example embodiments of FIGS. 1-4 depending on the shape, size, and/or other features of the vehicle in which the PAWU is disposed.

### 2. EXAMPLE HARDWARE AND SOFTWARE CONFIGURATIONS AND ARRANGEMENTS

FIG. 5 illustrates an example computing system 500, in accordance with various embodiments. The system 500 may include any combinations of the components as shown, which may be implemented as integrated circuits (ICs) or portions thereof, discrete electronic devices, or other modules, logic, hardware, software, firmware, middleware or a combination thereof adapted in the system 500, or as components otherwise incorporated within a chassis of a larger system, such as a vehicle 10 and/or CU 101. Additionally or alternatively, some or all of the components of system 500 may be combined and implemented as a suitable System-on-Chip (SoC), System-in-Package (SiP), multi-chip package (MCP), or some other like package. The system 500 is an embedded system or any other type of computer device discussed herein. In another example, the system 500 may be a separate and dedicated and/or special-purpose computer device designed specifically to carry out air-barrier solutions of the embodiments discussed herein.

The processor circuitry 502 comprises one or more processing elements/devices configurable to perform basic arithmetical, logical, and input/output operations by carrying out and/or executing instructions. According to various embodiments, processor circuitry 502 is configurable to perform some or all of the calculations associated with the preparation and/or generation of virtual graphics and/or other types of information that are to be projected by HUD system 1000 for display, in real time. Additionally, processor circuitry 502 is configurable to gather information from sensor circuitry 520 (e.g., process a video feed from a camera system or image capture devices), obtain user input from one or more I/O devices 586, and obtain vehicle input in substantially in real time. Some or all of the inputs may be received and/or transmitted via communication circuitry 509. In order to perform the aforementioned functions, the processor circuitry 502 may execute instructions 580, and/or may be loaded with an appropriate bit stream or logic blocks to generate virtual graphics based, at least in part, on any number of parameters, including, for example, input from sensor circuitry 520, input from I/O devices 586, input from actuators 522, input from ECUs 523, input from positioning circuitry 545, and/or the like. Additionally, processor circuitry 502 may be configurable to receive audio input, or to output audio, over an audio device 520. For example, processor circuitry 502 may be configurable to provide signals/commands to an audio output device 586 to provide audible instructions to accompany the displayed navigational route information or to provide audible alerts.

The processor circuitry 502 includes circuitry such as, but not limited to one or more processor cores and one or more of cache memory, low drop-out voltage regulators (LDOs), interrupt controllers, serial interfaces such as serial peripheral interface (SPI), inter-integrated circuit (I2C) or universal programmable serial interface circuit, real time clock (RTC), timer-counters including interval and watchdog timers, general purpose input-output (I/O), memory card controllers, interconnect (IX) controllers and/or interfaces, universal serial bus (USB) interfaces, mobile industry processor interface (MIPI) interfaces, Joint Test Access Group (JTAG) test access ports, and the like. The processor circuitry 502 may include on-chip memory circuitry or cache memory circuitry, which may include any suitable volatile and/or non-volatile memory, such as DRAM, SRAM, EPROM, EEPROM, Flash memory, solid-state memory, and/or any other type of memory device technology, such as those discussed herein.

The processor(s) of processor circuitry 502 may be, for example, one or more application processors or central processing units (CPUs), one or more graphics processing units (GPUs), one or more reduced instruction set computing (RISC) processors, one or more Acorn RISC Machine (ARM) processors, one or more complex instruction set computing (CISC) processors, one or more DSPs, one or more microprocessor without interlocked pipeline stages (MIPS), one or more programmable logic devices (PLDs) and/or hardware accelerators) such as field-programmable gate arrays (FPGAs), structured/programmable Application Specific Integrated Circuit (ASIC), programmable SoCs (PSoCs), etc., one or more microprocessors or controllers, or any suitable combination thereof. In some embodiments, the processor circuitry 502 may be implemented as a standalone system/device/package or as part of an existing system/device/package (e.g., an ECU/ECM, EEMS, etc.) of the vehicle 10. In some embodiments, the processor circuitry 502 may include special-purpose processor/controller to operate according to the various embodiments herein.

Individual processors (or individual processor cores) of the processor circuitry 502 may be coupled with or may include memory/storage and may be configurable to execute instructions stored in the memory/storage to enable various applications or operating systems to run on the system 500. In these embodiments, one or more processors (or cores) of the processor circuitry 502 may correspond to the processor 312 of FIG. 11 and is/are configurable to operate application software (e.g., HUD app) to provide specific services to a user of the system 500. In some embodiments, one or more processors (or cores) of the processor circuitry 502, such as one or more GPUs or GPU cores, may correspond to the HUD processor 1110 and is/are configurable to generate and render graphics as discussed previously.

As examples, the processor circuitry 502 may include an Intel® Architecture Core™ based processor, such as a Quark™, an Atom™, an i3, an i5, an i7, or an MCU-class processor, Pentium® processor(s), Xeon® processor(s), or another such processor available from Intel® Corporation, Santa Clara, California. However, any number other processors may be used, such as one or more of Advanced Micro Devices (AMD) Zen® Core Architecture, such as Ryzen® or EPYC® processor(s), Accelerated Processing Units (APUs), MxGPUs, Epyc® processor(s), or the like; A5-A12 and/or S1-S4 processor(s) from Apple® Inc., Snapdragon™ or Centrig™ processor(s) from Qualcomm® Technologies, Inc., Texas Instruments, Inc.® Open Multimedia Applications Platform (OMAP)™ processor(s); a MIPS-based design from MIPS Technologies, Inc. such as MIPS Warrior M-class, Warrior I-class, and Warrior P-class processors; an ARM-based design licensed from ARM Holdings, Ltd., such as the ARM Cortex-A, Cortex-R, and Cortex-M family of processors; the ThunderX2® provided by Cavium™, Inc.; or the like. Other examples of the processor circuitry 502 are mentioned elsewhere in the present disclosure.

In some implementations, the processor circuitry 502 may include a sensor hub, which acts as a coprocessor by processing data obtained from the sensor circuitry 520. The sensor hub may include circuitry configurable to integrate data obtained from each of the sensor circuitry 520 by performing arithmetical, logical, and input/output operations. In embodiments, the sensor hub may capable of timestamping obtained sensor data, providing sensor data to the processor circuitry 502 in response to a query for such data, buffering sensor data, continuously streaming sensor data to the processor circuitry 502 including independent streams for each sensor circuitry 520, reporting sensor data based upon predefined thresholds or conditions/triggers, and/or other like data processing functions.

The memory circuitry 504 comprises any number of memory devices arranged to provide primary storage from which the processor circuitry 502 continuously reads instructions 582 stored therein for execution. In some embodiments, the memory circuitry 504 includes on-die memory or registers associated with the processor circuitry 502. As examples, the memory circuitry 504 may include volatile memory such as random access memory (RAM), static RAM (SRAM), dynamic RAM (DRAM), synchronous DRAM (SDRAM), etc. The memory circuitry 504 may also include non-volatile memory (NVM) such as read-only memory (ROM), high-speed electrically erasable memory (commonly referred to as “flash memory”), and non-volatile RAM such as phase change memory, resistive memory such as magnetoresistive random access memory (MRAM), etc.

In some implementations, the processor circuitry 502 and memory circuitry 504 (and/or storage device 508) may comprise logic blocks or logic fabric, memory cells, input/output (I/O) blocks, and other interconnected resources that may be programmed to perform various functions of the example embodiments discussed herein. The memory cells may be used to store data in lookup-tables (LUTs) that are used by the processor circuitry 502 to implement various logic functions. The memory cells may include any combination of various levels of memory/storage including, but not limited to, EPROM, EEPROM, flash memory, SRAM, anti-fuses, etc. The memory circuitry 504 may also comprise persistent storage devices, which may be temporal and/or persistent storage of any type, including, but not limited to, non-volatile memory, optical, magnetic, and/or solid state mass storage, and so forth.

Storage circuitry 508 is arranged to provide (with shared or respective controllers) persistent storage of information such as data, applications, operating systems, and so forth. As examples, the storage circuitry 508 may be implemented as hard disk drive (HDD), a micro HDD, a solid-state disk drive (SSDD), flash memory, flash memory cards (e.g., SD cards, microSD cards, xD picture cards, and the like), USB flash drives, resistance change memories, phase change memories, holographic memories, or chemical memories, and the like. In an example, the storage circuitry 508 may be or may include memory devices that use chalcogenide glass, multi-threshold level NAND flash memory, NOR flash memory, single or multi-level Phase Change Memory (PCM), a resistive memory, nanowire memory, ferroelectric transistor random access memory (FeTRAM), anti-ferroelectric memory, magnetoresistive random access memory (MRAM) memory that incorporates memristor technology, phase change RAM (PRAM), resistive memory including the metal oxide base, the oxygen vacancy base and the conductive bridge Random Access Memory (CB-RAM), or spin transfer torque (STT)-MRAM, a spintronic magnetic junction memory based device, a magnetic tunneling junction (MTJ) based device, a Domain Wall (DW) and Spin Orbit Transfer (SOT) based device, a thyristor based memory device, or a combination of any of the above, or other memory. As shown, the storage circuitry 508 is included in the system 500; however, in other embodiments, storage circuitry 508 may be implemented as one or more separate devices that are mounted in vehicle 10 separate from the other elements of system 500.

The storage circuitry 508 is configurable to store computational logic 583 (or “modules 583”) in the form of software, firmware, microcode, or hardware-level instructions to implement the techniques described herein. The computational logic 583 may be employed to store working copies and/or permanent copies of programming instructions for the operation of various components of system 500 (e.g., drivers, libraries, application programming interfaces (APIs), etc.), an OS of system 500, one or more applications, and/or for carrying out the embodiments discussed herein. In some embodiments, the computational logic 583 may include one or more program code or other sequence of instructions for controlling the various components of the system 100 as discussed previously. The permanent copy of the programming instructions may be placed into persistent storage devices of storage circuitry 508 in the factory or in the field through, for example, a distribution medium (not shown), through a communication interface (e.g., from a distribution server (not shown)), or over-the-air (OTA). The computational logic 583 may be stored or loaded into memory circuitry 504 as instructions 582, which are then accessed for execution by the processor circuitry 502 to carry out the functions described herein. The instructions 582 direct the processor circuitry 502 to perform a specific sequence or flow of actions, for example, as described with respect to the flowchart(s) and block diagram(s) of operations and functionality depicted herein. The modules/logic 583 and/or instructions 580 may be implemented by assembler instructions supported by processor circuitry 502 or high-level languages that may be compiled into instructions 580 to be executed by the processor circuitry 502.

The computer program code for carrying out operations of the present disclosure (e.g., computational logic 583, instructions 582, 580, etc.) may be written in any combination of one or more programming languages, including an object oriented programming language such as Python, Ruby, Scala, Smalltalk, Java™, C++, C#, or the like; a procedural programming languages, such as the “C” programming language, the Go (or “Golang”) programming language, or the like; a scripting language such as JavaScript, Server-Side JavaScript (SSJS), PHP, Pearl, Python, Ruby or Ruby on Rails, Accelerated Mobile Pages Script (AMPscript), VBScript, and/or the like; a markup language such as HTML, XML, wiki markup or Wikitext, Wireless Markup Language (WML), etc.; a data interchange format/definition such as Java Script Object Notion (JSON), Apache® MessagePack™, etc.; a stylesheet language such as Cascading Stylesheets (CSS), extensible stylesheet language (XSL), or the like; an interface definition language (IDL) such as Apache® Thrift, Abstract Syntax Notation One (ASN.1), Google® Protocol Buffers (protobuf), etc.; or some other suitable programming languages including proprietary programming languages and/or development tools, or any other languages or tools as discussed herein. The computer program code for carrying out operations of the present disclosure may also be written in any combination of the programming languages discussed herein. The program code may execute entirely on the system 500, partly on the system 500 as a stand-alone software package, partly on the system 500 and partly on a remote computer, or entirely on the remote computer. In the latter scenario, the remote computer may be connected to the system 500 through any type of network (e.g., network 517).

The OS of system 500 manages computer hardware and software resources, and provides common services for various applications (e.g., application 110). The OS of system 500 may be or include the on-board operating system 1120 discussed previously. The OS may include one or more drivers or APIs that operate to control particular devices that are embedded in the system 500, attached to the system 500, or otherwise communicatively coupled with the system 500. The drivers may include individual drivers allowing other components of the system 500 to interact or control various I/O devices that may be present within, or connected to, the system 500. For example, the drivers may include a display driver (or HUD system driver) to control and allow access to the HUD system 1000, a touchscreen driver to control and allow access to a touchscreen interface of the system 500, sensor drivers to obtain sensor readings of sensor circuitry 520 and control and allow access to sensor circuitry 520, actuator drivers to obtain actuator positions of the actuators 524 and/or control and allow access to the actuators 522, ECU drivers to obtain control system information from one or more of the ECUs 523, audio drivers to control and allow access to one or more audio devices. The OSs may also include one or more libraries, drivers, APIs, firmware, middleware, software glue, etc., which provide program code and/or software components for one or more applications to obtain and use the data from other applications operated by the system 500.

In some embodiments, the OS may be a general purpose OS, while in other embodiments, the OS is specifically written for and tailored to the system 500. For example, the OS may be Unix or a Unix-like OS such as Linux e.g., provided by Red Hat Enterprise, Windows 10™ provided by Microsoft Corp.®, macOS provided by Apple Inc.®, or the like. In another example, the OS may be a mobile OS, such as Android® provided by Google Inc.®, iOS® provided by Apple Inc.®, Windows 10 Mobile® provided by Microsoft Corp.®, KaiOS provided by KaiOS Technologies Inc., or the like. In another example, the OS may be an embedded OS or a real-time OS (RTOS), such as Windows Embedded Automotive provided by Microsoft Corp.®, Windows 10 For IoT® provided by Microsoft Corp.®, Apache Mynewt provided by the Apache Software Foundation®, Micro-Controller Operating Systems (“MicroC/OS” or “μC/OS”) provided by Micrium®, Inc., FreeRTOS, VxWorks® provided by Wind River Systems, Inc.®, PikeOS provided by Sysgo AG®, Android Things® provided by Google Inc.®, QNX® RTOS provided by BlackBerry Ltd., or any other suitable embedded OS or RTOS, such as those discussed herein. In another example, the OS may be a robotics middleware framework, such as Robot Operating System (ROS), Robotics Technology (RT)-middleware provided by Object Management Group®, Yet Another Robot Platform (YARP), and/or the like.

In embodiments where the processor circuitry 502 and memory circuitry 504 includes hardware accelerators in addition to or alternative to processor cores, the hardware accelerators may be pre-configured (e.g., with appropriate bit streams, logic blocks/fabric, etc.) with the logic to perform some functions of the embodiments herein (in lieu of employment of programming instructions to be executed by the processor core(s)). In one example, the processor circuitry 502, memory circuitry 504, and/or storage circuitry 508 may be packaged together in a suitable SoC or the like and may operate as the CU 101 as discussed previously.

The components of system 500 and/or vehicle 10 communicate with one another over an interconnect (IX) 506. In various embodiments, IX 506 is a controller area network (CAN) bus system, a Time-Trigger Protocol (TTP) system, or a FlexRay system, which may allow various devices (e.g., ECUs 523, sensor circuitry 520, actuators 522, etc.) to communicate with one another using messages or frames. Additionally or alternatively, the IX 506 may include any number of other IX technologies, such as a Local Interconnect Network (LIN), industry standard architecture (ISA), extended ISA (EISA), inter-integrated circuit (I2C), a serial peripheral interface (SPI), point-to-point interfaces, power management bus (PMBus), peripheral component interconnect (PCI), PCI express (PCIe), Ultra Path Interface (UPI), Accelerator Link (IAL), Common Application Programming Interface (CAPI), QuickPath Interconnect (QPI), Omni-Path Architecture (OPA) IX, RapidIO™ system interconnects, Ethernet, Cache Coherent Interconnect for Accelerators (CCIA), Gen-Z Consortium IXs, Open Coherent Accelerator Processor Interface (OpenCAPI), and/or any number of other IX technologies. The IX 506 may be a proprietary bus, for example, used in a SoC based system.

The communication circuitry 509 is a hardware element, or collection of hardware elements, used to communicate over one or more networks (e.g., network 517) and/or with other devices. The communication circuitry 509 includes modem 510 and transceiver circuitry (“TRx”) 512. The modem 510 includes one or more processing devices (e.g., baseband processors) to carry out various protocol and radio control functions. Modem 510 interfaces with application circuitry of system 500 (e.g., a combination of processor circuitry 502 and memory 504) for generation and processing of baseband signals and for controlling operations of the TRx 512. The modem 510 handles various radio control functions that enable communication with one or more radio networks 517 via the TRx 512 according to one or more wireless communication protocols, such as those discussed herein. The modem 510 may include circuitry such as, but not limited to, one or more single-core or multi-core processors (e.g., one or more baseband processors) or control logic to process baseband signals received from a receive signal path of the TRx 512, and to generate baseband signals to be provided to the TRx 512 via a transmit signal path. In various embodiments, the modem 510 may implement a real-time OS (RTOS) to manage resources of the modem 510, schedule tasks, etc.

The communication circuitry 509 also includes TRx 512 to enable communication with wireless networks 517 using modulated electromagnetic radiation through a non-solid medium. TRx 512 includes a receive signal path, which comprises circuitry to convert analog RF signals (e.g., an existing or received modulated waveform) into digital baseband signals to be provided to the modem 510. The TRx 512 also includes a transmit signal path, which comprises circuitry configurable to convert digital baseband signals provided by the modem 510 to be converted into analog RF signals (e.g., modulated waveform) that will be amplified and transmitted via an antenna array including one or more antenna elements (not shown). The antenna array is coupled with the TRx 512 using metal transmission lines or the like. The antenna array may be a one or more microstrip antennas or printed antennas that are fabricated on the surface of one or more printed circuit boards; a patch antenna array formed as a patch of metal foil in a variety of shapes; a glass-mounted antenna array or “on-glass” antennas; or some other known antenna or antenna elements.

The TRx 512 may include one or more radios that are compatible with, and/or may operate according to any one or more of the following radio communication technologies and/or standards including but not limited to: a Global System for Mobile Communications (GSM) radio communication technology, a General Packet Radio Service (GPRS) radio communication technology, an Enhanced Data Rates for GSM Evolution (EDGE) radio communication technology, and/or a 3GPP radio communication technology such as Universal Mobile Telecommunications System (UMTS), Freedom of Multimedia Access (FOMA), Code Division Multiple Access 2000 (CDM2000), Cellular Digital Packet Data (CDPD), Mobitex, Circuit Switched Data (CSD), High-Speed Circuit-Switched Data (HSCSD), UMTS Wideband Code Division Multiple Access, High Speed Packet Access (HSPA), High-Speed Downlink Packet Access (HSDPA), High-Speed Uplink Packet Access (HSUPA), High Speed Packet Access Plus (HSPA+), UMTS-Time-Division Duplex (UMTS-TDD), Time Division-Code Division Multiple Access (TD-CDMA), Time Division-Synchronous Code Division Multiple Access (TD-CDMA), Long Term Evolution (LTE), LTE-Advanced (LTE-A), LTE Extra, LTE-A Pro, LTE Licensed-Assisted Access (LAA), MuLTEfire, UMTS Terrestrial Radio Access (UTRA), Evolved UMTS Terrestrial Radio Access (E-UTRA), Fifth Generation (5G) or New Radio (NR), 3GPP device-to-device (D2D) or Proximity Services (ProSe), 3GPP cellular vehicle-to-everything (V2X), Evolution-Data Optimized or Evolution-Data Only (EV-DO), Advanced Mobile Phone System (AMPS), Total Access Communication System/Extended Total Access Communication System (TACS/ETACS), Digital AMPS (D-AMPS), Push-to-talk (PTT), Mobile Telephone System (MTS), Improved Mobile Telephone System (IMTS), Advanced Mobile Telephone System (AMTS), Offentlig Landmobil Telefoni (OLT) which is Norwegian for Public Land Mobile Telephony, Mobiltelefonisystem D (MTD) which is Swedish for Mobile telephony system D, Public Automated Land Mobile (Autotel/PALM), Autoradiopuhelin (ARP) which is Finnish for “car radio phone”, Nordic Mobile Telephony (NMT), Nippon Telegraph and Telephone (NTT), High capacity (Hicap) version of NTT, Cellular Digital Packet Data (CDPD), Mobitex, DataTAC, Integrated Digital Enhanced Network (iDEN), Personal Digital Cellular (PDC), Circuit Switched Data (CSD), Personal Handy-phone System (PHS), Wideband Integrated Digital Enhanced Network (WiDEN), iBurst, Unlicensed Mobile Access (UMA) also referred to as also referred to as 3GPP Generic Access Network (GAN), Bluetooth®, Bluetooth Low Energy (BLE), IEEE 802.15.4 based protocols (e.g., IPv6 over Low power Wireless Personal Area Networks (6LoWPAN), WirelessHART, MiWi, Thread, I600.11a, etc.) WiFi-direct, ANT/ANT+, ZigBee, Z-Wave, Universal Plug and Play (UPnP), Low-Power Wide-Area-Network (LPWAN), LoRaWAN™ (Long Range Wide Area Network), Sigfox, Wireless Gigabit Alliance (WiGig) standard, mmWave standards in general (wireless systems operating at 10-300 GHz and above such as WiGig, IEEE 802.11ad, IEEE 802.11ay, etc.), technologies operating above 300 GHz and THz bands, (3GPP/LTE based or IEEE 802.11p and other), Dedicated Short Range Communications (DSRC) communication systems such as Intelligent-Transport-Systems and others, the European ITS-G5 system (i.e. the European flavor of IEEE 802.11p based DSRC, including ITS-G5A (i.e., Operation of ITS-G5 in European ITS frequency bands dedicated to ITS for safety related applications in the frequency range 5,875 GHz to 5,905 GHz), ITS-G5B (i.e., Operation in European ITS frequency bands dedicated to ITS non-safety applications in the frequency range 5,855 GHz to 5,875 GHz), ITS-G5C (i.e., Operation of ITS applications in the frequency range 5,470 GHz to 5,725 GHz)), etc. In addition to the aforementioned standards, any number of satellite uplink technologies may be used for the TRx 512 including, for example, radios compliant with standards issued by the International Telecommunication Union (ITU), or the European Telecommunications Standards Institute (ETSI), among others, both existing and not yet formulated.

Network interface circuitry/controller (NIC) 516 may be included to provide wired communication to the network 517 or to other devices using a standard network interface protocol. In most cases, the NIC 516 may be used to transfer data over a network (e.g., network 517) via a wired connection while the vehicle is stationary (e.g., in a garage, testing facility, or the like). The standard network interface protocol may include Ethernet, Ethernet over GRE Tunnels, Ethernet over Multiprotocol Label Switching (MPLS), Ethernet over USB, or may be based on other types of network protocols, such as Controller Area Network (CAN), Local Interconnect Network (LIN), DeviceNet, ControlNet, Data Highway+, PROFIBUS, or PROFINET, among many others. Network connectivity may be provided to/from the system 500 via NIC 516 using a physical connection, which may be electrical (e.g., a “copper interconnect”) or optical. The physical connection also includes suitable input connectors (e.g., ports, receptacles, sockets, etc.) and output connectors (e.g., plugs, pins, etc.). The NIC 516 may include one or more dedicated processors and/or FPGAs to communicate using one or more of the aforementioned network interface protocols. In some implementations, the NIC 516 may include multiple controllers to provide connectivity to other networks using the same or different protocols. For example, the system 500 may include a first NIC 516 providing communications to the cloud over Ethernet and a second NIC 516 providing communications to other devices over another type of network.

The input/output (I/O) interface 518 is configurable to connect or coupled the system 500 with external devices or subsystems. The external interface 518 may include any suitable interface controllers and connectors to couple the system 500 with the external components/devices, such as an external expansion bus (e.g., Universal Serial Bus (USB), FireWire, PCIe, Thunderbolt, Lighting™, etc.), used to connect system 500 with external components/devices, such as sensor circuitry 520, actuators 522, electronic control units (ECUs) 523, positioning system 545, input device(s) 586, and picture generation units (PGUs) 530. In some cases, the I/O interface circuitry 518 may be used to transfer data between the system 500 and another computer device (e.g., a laptop, a smartphone, or some other user device) via a wired connection. I/O interface circuitry 518 may include any suitable interface controllers and connectors to interconnect one or more of the processor circuitry 502, memory circuitry 504, storage circuitry 508, communication circuitry 509, and the other components of system 500. The interface controllers may include, but are not limited to, memory controllers, storage controllers (e.g., redundant array of independent disk (RAID) controllers, baseboard management controllers (BMCs), input/output controllers, host controllers, etc. The connectors may include, for example, busses (e.g., IX 506), ports, slots, jumpers, interconnect modules, receptacles, modular connectors, etc. The I/O interface circuitry 518 may also include peripheral component interfaces including, but are not limited to, non-volatile memory ports, USB ports, audio jacks, power supply interfaces, on-board diagnostic (OBD) ports, etc.

The sensor circuitry 520 includes devices, modules, or subsystems whose purpose is to detect events or changes in its environment and send the information (sensor data) about the detected events to some other a device, module, subsystem, etc. Examples of such sensors 520 include, inter alia, inertia measurement units (IMU) comprising accelerometers, gyroscopes, and/or magnetometers; microelectromechanical systems (MEMS) or nanoelectromechanical systems (NEMS) comprising 3-axis accelerometers, 3-axis gyroscopes, and/or magnetometers; level sensors; flow sensors; temperature sensors (e.g., thermistors); pressure sensors; barometric pressure sensors; gravimeters; altimeters; image capture devices (e.g., cameras); light detection and ranging (LiDAR) sensors; proximity sensors (e.g., infrared radiation detector and the like), depth sensors, ambient light sensors, ultrasonic transceivers; microphones; etc. In some embodiments, sensor circuitry 520 may correspond to a gauge/sensor 103, sensor 105, and/or flow meter 109B as discussed previously.

Some of the sensor circuitry 520 may be sensors used for various vehicle control systems, and may include, inter alia, exhaust sensors including exhaust oxygen sensors to obtain oxygen data and manifold absolute pressure (MAP) sensors to obtain manifold pressure data; mass air flow (MAF) sensors to obtain intake air flow data; intake air temperature (IAT) sensors to obtain IAT data; ambient air temperature (AAT) sensors to obtain AAT data; ambient air pressure (AAP) sensors to obtain AAP data; catalytic converter sensors including catalytic converter temperature (CCT) to obtain CCT data and catalytic converter oxygen (CCO) sensors to obtain CCO data; vehicle speed sensors (VSS) to obtain VSS data; exhaust gas recirculation (EGR) sensors including EGR pressure sensors to obtain ERG pressure data and EGR position sensors to obtain position/orientation data of an EGR valve pintle; Throttle Position Sensor (TPS) to obtain throttle position/orientation/angle data; a crank/cam position sensors to obtain crank/cam/piston position/orientation/angle data; coolant temperature sensors; and/or other like sensors embedded in vehicle 10. The sensor circuitry 520 may include other sensors such as an accelerator pedal position sensor (APP), accelerometers, magnetometers, level sensors, flow/fluid sensors, barometric pressure sensors, and the like.

The positioning circuitry 545 includes circuitry to receive and decode signals transmitted/broadcasted by a positioning network of a global navigation satellite system (GNSS). Examples of navigation satellite constellations (or GNSS) include United States' Global Positioning System (GPS), Russia's Global Navigation System (GLONASS), the European Union's Galileo system, China's BeiDou Navigation Satellite System, a regional navigation system or GNSS augmentation system (e.g., Navigation with Indian Constellation (NAVIC), Japan's Quasi-Zenith Satellite System (QZSS), France's Doppler Orbitography and Radio-positioning Integrated by Satellite (DORIS), etc.), or the like. The positioning circuitry 545 comprises various hardware elements (e.g., including hardware devices such as switches, filters, amplifiers, antenna elements, and the like to facilitate OTA communications) to communicate with components of a positioning network, such as navigation satellite constellation nodes. In some embodiments, the positioning circuitry 545 may include a Micro-Technology for Positioning, Navigation, and Timing (Micro-PNT) IC that uses a master timing clock to perform position tracking/estimation without GNSS assistance. The positioning circuitry 545 may also be part of, or interact with, the communication circuitry 509 to communicate with the nodes and components of the positioning network. The positioning circuitry 545 may also provide position data and/or time data to the application circuitry, which may use the data to synchronize operations with various infrastructure (e.g., radio base stations), for turn-by-turn navigation, or the like. Additionally or alternatively, the positioning circuitry 545 may be incorporated in, or work in conjunction with the communication circuitry to determine the position or location of the vehicle 10 by, for example, implementing the LTE Positioning Protocol (LPP), Wi-Fi positioning system (WiPS or WPS) methods, triangulation, signal strength calculations, and/or some other suitable localization technique(s).

Individual ECUs 523 may be embedded systems or other like computer devices that control a corresponding system of the vehicle 10. In embodiments, individual ECUs 523 may each have the same or similar components as the system 500, such as a microcontroller or other like processor device, memory device(s), communications interfaces, and the like. In embodiments, the ECUs 523 may include, inter alia, a Drivetrain Control Unit (DCU), an Engine Control Unit (ECU), an Engine Control Module (ECM), EEMS, a Powertrain Control Module (PCM), a Transmission Control Module (TCM), a Brake Control Module (BCM) including an anti-lock brake system (ABS) module and/or an electronic stability control (ESC) system, a Central Control Module (CCM), a Central Timing Module (CTM), a General Electronic Module (GEM), a Body Control Module (BCM), a Suspension Control Module (SCM), a Door Control Unit (DCU), a Speed Control Unit (SCU), a Human-Machine Interface (HMI) unit, a Telematic Control Unit (TTU), a Battery Management System (which may be the same or similar as battery monitor 526) and/or any other entity or node in a vehicle system. In some embodiments, the one or more of the ECUs 523 and/or system 500 may be part of or included in a Portable Emissions Measurement Systems (PEMS).

The actuators 522 are devices that allow system 500 to change a state, position, orientation, move, and/or control a mechanism or system in the vehicle 10. The actuators 522 comprise electrical and/or mechanical devices for moving or controlling a mechanism or system, and converts energy (e.g., electric current or moving air and/or liquid) into some kind of motion. The actuators 522 may include one or more electronic (or electrochemical) devices, such as piezoelectric biomorphs, solid state actuators, solid state relays (SSRs), shape-memory alloy-based actuators, electroactive polymer-based actuators, relay driver integrated circuits (ICs), and/or the like. The actuators 522 may include one or more electromechanical devices such as pneumatic actuators, hydraulic actuators, electromechanical switches including electromechanical relays (EMRs), motors (e.g., linear motors, DC motors, brushless motors, stepper motors, servomechanisms, ultrasonic piezo motor with optional position feedback, screw-type motors, etc.), mechanical gears, magnetic switches, valve actuators, fuel injectors, ignition coils, wheels, thrusters, propellers, claws, clamps, hooks, an audible sound generator, and/or other like electromechanical components. As examples, the translation device or motor 1180 discussed previously may be among the one or more of the actuators 522. The system 500 may be configurable to operate one or more actuators 522 based on one or more captured events and/or instructions or control signals received from various ECUs 523 or system 500. In embodiments, the system 500 may transmit instructions to various actuators 522 (or controllers that control one or more actuators 522) to reconfigure an electrical network as discussed herein. In various embodiments, some of the actuators 522 correspond to the pressure generator 104, decontamination device 107, and/or the flow regulator 109 as discussed previously.

In embodiments, system 500 and/or ECUs 523 are configurable to operate one or more actuators 522 by transmitting/sending instructions or control signals to one or more actuators 522 based on detected events. Individual ECUs 523 may be capable of reading or otherwise obtaining sensor data from the sensor circuitry 520, processing the sensor data to generate control system data, and providing the control system data to the system 500 for processing. The control system information may be a type of state information discussed previously. For example, an ECU 523 may provide engine revolutions per minute (RPM) of an engine of the vehicle 10, fuel injector activation timing data of one or more cylinders and/or one or more injectors of the engine, ignition spark timing data of the one or more cylinders (e.g., an indication of spark events relative to crank angle of the one or more cylinders), transmission gear ratio data and/or transmission state data (which may be supplied to the ECU 523 by the TCU), real-time calculated engine load values from the ECM, etc.; a TCU may provide transmission gear ratio data, transmission state data, etc.; and the like.

The I/O devices 586 may be present within, or connected to, the system 500. The I/O devices 586 include input devices and output devices including one or more user interfaces designed to enable user interaction with the system 500 and/or peripheral component interaction with the system 500 via peripheral component interfaces. The input devices include any physical or virtual means for accepting an input including, inter alia, one or more physical or virtual buttons (e.g., a reset button), a physical keyboard, keypad, mouse, touchpad, touchscreen, microphones, scanner, headset, and/or the like. It should be noted that user input may comprise voice commands, control input (e.g., via buttons, knobs, switches, etc.), an interface with a smartphone, or any combination thereof.

The output devices are used to show or convey information, such as sensor readings, actuator position(s), or other like information. Data and/or graphics may be displayed on one or more user interface components of the output devices. The output devices may include any number and/or combinations of audio or visual display, including, inter alia, one or more simple visual outputs/indicators (e.g., binary status indicators (e.g., light emitting diodes (LEDs)) and multi-character visual outputs, or more complex outputs such as display devices or touchscreens (e.g., Liquid Chrystal Displays (LCD), LED displays, quantum dot displays, projectors, Head-Up Display (HUD) devices, etc.), with the output of characters, graphics, multimedia objects, and the like being generated or produced from the operation of the system 500. The output devices may also include speakers or other audio emitting devices, printer(s), and/or the like. In some embodiments, the sensor circuitry 520 may be used as an input device (e.g., an image capture device, motion capture device, or the like) and one or more actuators 522 may be used as an output device (e.g., an actuator to provide haptic feedback or the like). In another example, near-field communication (NFC) circuitry comprising an NFC controller coupled with an antenna element and a processing device may be included as an input device to read electronic tags and/or connect with another NFC-enabled device.

The battery 524a and/or power block 524b may power the system 500. In embodiments, the battery 524a may be a typical lead-acid automotive battery, although in some embodiments, such as when vehicle 10 is a hybrid vehicle, the battery 524a may be a lithium ion battery, a metal-air battery, such as a zinc-air battery, an aluminum-air battery, a lithium-air battery, a lithium polymer battery, and the like. The battery monitor 526 may be included in the system 500 to track/monitor various parameters of the battery 524a, such as a state of charge (SoCh) of the battery 524, state of health (SoH), and the state of function (SoF) of the battery 524. The battery monitor 526 may include a battery monitoring IC, which may communicate battery information to the processor circuitry 502 over the IX 506.

While not shown, various other devices may be present within, or connected to, the system 500. For example, I/O devices, such as a display, a touchscreen, or keypad may be connected to the system 500 via IX 506 to accept input and display outputs. In another example, GNSS and/or GPS circuitry and associated applications may be included in or connected with system 500 to determine a geolocation of the vehicle 10. In another example, the communication circuitry 1205 may include a Universal Integrated Circuit Card (UICC), embedded UICC (eUICC), and/or other elements/components that may be used to communicate over one or more wireless networks 517.

### 3. EXAMPLE IMPLEMENTATIONS

Some non-limiting example as provided infra. The following examples pertain to further embodiments. Specifics in the examples may be used anywhere in one or more embodiments. All optional features of the apparatus(es) described herein may also be implemented with respect to a method or process.

Example 1 includes protective air wall unit (PAWU) for use in an enclosed space, the PAWU comprising: pressure generation means for generating an airflow; decontamination means coupled to the pressure generation means; and control means communicatively coupled with the pressure generation means, the control means for controlling the pressure generation means to generate the airflow to flow through the enclosed space such that an air barrier is created that prevents a contaminate from crossing the air barrier and carries the contaminate to the decontamination means, and the decontamination means for reducing or preventing the contaminate from recirculating through the enclosed space.

Example 2 includes the PAWU of example 1 and/or some other example(s) herein, wherein the pressure generation means comprises an airflow output and an airflow input, and the decontamination means coupled to the airflow input of the pressure generation means, and wherein the airflow recirculates to the pressure generation means via the airflow input after the decontamination means substantially removes the contaminate.

Example 3 includes the PAWU of examples 1-2, and/or some other example(s) herein wherein the PAWU is coupled to a first end of a first duct and a first end of a second duct, and the control means is further for controlling the pressure generation means to generate the airflow to flow through the first duct, out of a first nozzle coupled to a second end of the first duct, and through a second nozzle coupled to a second end of the second duct, wherein the air barrier is created between the first nozzle and the second nozzle.

Example 4 includes the PAWU of examples 1-3 and/or some other example(s) herein, further comprising: first sensing means for measuring a pressure level of the airflow output by the pressure generation means; and second sensing means for measuring a quality or operation of the decontamination means.

Example 5 includes the PAWU of example 4 and/or some other example(s) herein, wherein the decontamination means comprises an air filter, wherein the air filter is one or more of a paper filter, polyurethane foam filter, cotton filter, stainless steel mesh filter, fiberglass filter, a high-efficiency particulate absorbing (HEPA) air filter, a semi-HEPA filter, an Ultra-Low Particulate Air (ULPA) filter, or a semi-ULPA filter.

Example 6 includes the PAWU of examples 4-5 and/or some other example(s) herein, wherein the decontamination means comprises an ultraviolet germicidal irradiation (UVGI) system.

Example 7 includes the PAWU of examples 4-6 and/or some other example(s) herein, wherein the decontamination means comprises an electrostatic precipitator (ESP) system.

Example 8(a) includes the PAWU of examples 4-7 and/or some other example(s) herein, wherein the first sensing means comprises pressure sensing means.

Example 8(b) includes the PAWU of examples 4-8(a) and/or some other example(s) herein, wherein the second sensing means comprises one or more of pressure sensing means, particle sensing means, microwave cavity sensing means, electrochemical nanosensing means, and/or plasmonic photothermal biosensing means.

Example 9 includes the PAWU of examples 4-8(b) and/or some other example(s) herein, wherein the control means is further for: obtaining one or both of first sensor data from the first sensor and second sensor data from the second sensor; and adjusting operational parameters of the pressure generation means based on the first sensor data or the second sensor data.

Example 10 includes the PAWU of any one of examples 1-9 and/or some other example(s) herein, further comprising flow regulation means for controlling the pressure of the airflow, wherein the flow regulation means comprises: restricting means for maintaining a set pressure; and measurement means for measuring the pressure of the airflow for the restricting element.

Example 11 includes the PAWU of any one of examples 1-10 and/or some other example(s) herein, wherein the pressure generation means comprises one or more of an air pump, an air compressor, a vacuum pump, a fan, or an air knife.

Example 12 includes the PAWU of any one of examples 1-11 and/or some other example(s) herein, wherein the enclosed space is a cabin of a vehicle.

Example 13 includes a vehicle, comprising: air circulating means; and protective air wall unit (PAWU) coupled with the air circulating means, the PAWU including: a pressure generation means for generating an airflow; decontamination means coupled to the pressure generation means; and control means communicatively coupled with the pressure generation means, the control means for controlling the pressure generation means to generate the airflow to flow through the enclosed space such that an air barrier is created that prevents a contaminate from crossing the air barrier and carries the contaminate to the decontamination means, and the decontamination means for reducing or preventing the contaminate from recirculating through the enclosed space.

Example 14 includes the vehicle of example 13 and/or some other example(s) herein, wherein the pressure generation means further comprises an airflow output means and an airflow input means, and the decontamination means is coupled to the airflow input means, and the airflow recirculates to the pressure generation means via the airflow input means after the decontamination means substantially removes the contaminate from the airflow.

Example 15 includes the vehicle of example 14 and/or some other example(s) herein, wherein the air circulating means comprises an air supply means and a recirculation means, the airflow output means is coupled to a first end of the air supply means, and the airflow input means is coupled to a first end of a recirculation means, and the control means is further for: controlling the pressure generation means to generate the airflow to flow through the air supply means, out of output means coupled to a second end of the air supply means, and through input means coupled to a second end of the recirculation means, wherein the air barrier is created between the input means and the output means.

Example 16 includes the vehicle of examples 13-15 and/or some other example(s) herein, further comprising: first sensing means for measuring a pressure level of the airflow output by the pressure generation means; and second sensing means configured to measure a quality or operation of the decontamination means.

Example 17 includes the vehicle of example 16 and/or some other example(s) herein, wherein the control means is further for: obtaining one or both of first sensor data from the first sensor and second sensor data from the second sensor; and adjusting operational parameters of the pressure generation means based on the first sensor data or the second sensor data.

Example 18 includes the vehicle of examples 16-17 and/or some other example(s) herein, wherein the decontamination means comprises one or more of an air filter, an ultraviolet germicidal irradiation (UVGI) system, and an electrostatic precipitator (ESP) system, and the pressure generation means comprises one or more of an air pump, an air compressor, a vacuum pump, a fan, or an air knife.

Example 19(a) includes the vehicle of examples 16-18 and/or some other example(s) herein, wherein the first sensing means comprises pressure sensing means.

Example 19(b) includes the vehicle of examples 16-19(b) and/or some other example(s) herein, wherein the second sensing means comprises one or more of pressure sensing means, particle sensing means, microwave cavity sensing means, electrochemical nanosensing means, and/or plasmonic photothermal biosensing means

Example 20 includes the vehicle of examples 13-19(b) and/or some other example(s) herein, further comprising: flow regulation means for controlling the pressure of the airflow, wherein the flow regulation means comprises: restricting means for maintaining a set pressure; and measurement means for measuring the pressure of the airflow for the restricting element.

Example 21 includes the vehicle of any one of examples 13-20 and/or some other example(s) herein, wherein the pressure generation means comprises one or more of an air pump, an air compressor, a vacuum pump, a fan, or an air knife.

Example 22 includes the vehicle of any one of examples 13-21 and/or some other example(s) herein, wherein the enclosed space is a cabin of the vehicle.

Example 23 includes a controller for operating a protective air wall unit (PAWU) for use in an enclosed space, the PAWU comprising a pressure generator configured to generate an airflow and a decontamination device coupled to the pressure generator configured to reduce or prevent the contaminate from recirculating through the enclosed space, the control unit comprising: processor circuitry configurable to determine operational parameters for generation of the airflow to flow through the enclosed space such that an air barrier is created that prevents a contaminate from crossing the air barrier and carries the contaminate to the decontamination device; and interface circuitry configurable to send control signaling indicating the determined operational parameters, the control signaling to control the pressure generator to generate the airflow.

Example 24 includes the controller of claim 23 and/or some other example(s) herein, wherein the PAWU is coupled to a first end of a first duct and a first end of a second duct, and the processor circuitry is further configurable to: control the pressure generator to generate an airflow to flow through the first duct, out of a first nozzle coupled to a second end of the first duct, and through a second nozzle coupled to a second end of the second duct, wherein the air barrier is created between the first nozzle and the second nozzle.

Example 25 includes the controller of claims 23-24 and/or some other example(s) herein, wherein the processor circuitry is further configurable to: obtain first sensor data from a first sensor, the first sensor data representative of measured pressure levels of the airflow output by the pressure generator; obtain second sensor data from a second sensor, the second sensor data representative of measured quality or operation of the decontamination device; and adjust the operational parameters based on the first sensor data or the second sensor data.

Example 26 includes the controller of claim 25 and/or some other example(s) herein, wherein the first sensor comprises a pressure sensor, and the second sensor comprises one or more of a pressure sensor, a particle sensor, a microwave cavity sensor, a electrochemical nanosensor, or a plasmonic photothermal biosensor.

Example 27 includes the controller of claims 23-26 and/or some other example(s) herein, wherein the decontamination device comprises an ultraviolet germicidal irradiation (UVGI) system or an electrostatic precipitator (ESP) system, and wherein: the processor circuitry is further configurable to determine other operational parameters for controlling the UVGI or the ESP system; and interface circuitry configurable to send the other control signaling indicating the determined other operational parameters, the other control signaling to control the UVGI or the ESP system.

Example 28 includes one or more computer-readable storage media comprising instructions, wherein execution of the instructions by a controller of a protective air wall unit (PAWU) is to cause the controller to operate according to any of examples 1-27 and/or some other example(s) herein.

### 4. TERMINOLOGY

For the purposes of the present document, the phrase “A or B” means (A), (B), or (A and B). The phrases “A/B” and “A or B” mean (A), (B), or (A and B), similar to the phrase “A and/or B.” For the purposes of the present disclosure, the phrase “at least one of A and B” means (A), (B), or (A and B). The description may use the terms “embodiment” or “embodiments,” which may each refer to one or more of the same or different embodiments. Furthermore, the terms “comprising,” “including,” “having,” and the like, as used with respect to one or more embodiments, are synonymous, are generally intended as “open” terms (e.g., the term “including” should be interpreted as “including but not limited to,” the term “having” should be interpreted as “having at least,” the term “includes” should be interpreted as “includes but is not limited to,” etc.), and specify the presence of stated features, integers, steps, operations, elements, and/or components, but do not preclude the presence or addition of one or more other features, integers, steps, operations, elements, components, and/or groups thereof. The terms “comprises,” “comprising,” “includes,” and/or “including,” The phrase “in various embodiments,” “in some embodiments,” and the like are used repeatedly. The phrase generally does not refer to the same embodiments; however, it may. The present disclosure may use the phrases “in an embodiment,” “in embodiments,” “in some embodiments,” and/or “in various embodiments,” which may each refer to one or more of the same or different embodiments.

The terms “coupled,” “communicatively coupled,” along with derivatives thereof are used herein. The term “coupled” may mean two or more elements are in direct physical or electrical contact with one another, may mean that two or more elements indirectly contact each other but still cooperate or interact with each other, and/or may mean that one or more other elements are coupled or connected between the elements that are said to be coupled with each other. The term “directly coupled” may mean that two or more elements are in direct contact with one another. The term “communicatively coupled” may mean that two or more elements may be in contact with one another by a means of communication including through a wire or other interconnect connection, through a wireless communication channel or ink, and/or the like.

The term “fabrication” refers to the creation of a metal structure using fabrication means. The term “fabrication means” as used herein refers to any suitable tool or machine that is used during a fabrication process and may involve tools or machines for cutting (e.g., using manual or powered saws, shears, chisels, routers, torches including handheld torches such as oxy-fuel torches or plasma torches, and/or computer numerical control (CNC) cutters including lasers, mill bits, torches, water jets, routers, etc.), bending (e.g., manual, powered, or CNC hammers, pan brakes, press brakes, tube benders, roll benders, specialized machine presses, etc.), assembling (e.g., by welding, soldering, brazing, crimping, coupling with adhesives, riveting, using fasteners, etc.), molding or casting (e.g., die casting, centrifugal casting, injection molding, extrusion molding, matrix molding, three-dimensional (3D) printing techniques including fused deposition modeling, selective laser melting, selective laser sintering, composite filament fabrication, fused filament fabrication, stereolithography, directed energy deposition, electron beam freeform fabrication, etc.), and PCB and/or semiconductor manufacturing techniques (e.g., silk-screen printing, photolithography, photoengraving, PCB milling, laser resist ablation, laser etching, plasma exposure, atomic layer deposition (ALD), molecular layer deposition (MLD), chemical vapor deposition (CVD), rapid thermal processing (RTP), and/or the like).

The term “fastener”, “fastening means”, or the like refers to device that mechanically joins or affixes two or more objects together, and may include threaded fasteners (e.g., bolts, screws, nuts, threaded rods, etc.), pins, linchpins, r-clips, clips, pegs, clamps, dowels, cam locks, latches, catches, ties, hooks, magnets, molded or assembled joineries, and/or the like.

The term “air flow” or “airflow” refers to the movement of air and/or a measurement of the amount of air per unit of time that flows through a particular device. The “airflow” can be induced through mechanical means or can take place passively, as a function of pressure differentials present in the environment. The “airflow” may be a laminar or turbulent flow pattern. A laminar airflow occurs when air can flow smoothly, and exhibits a parabolic velocity profile. A turbulent airflow occurs when there is an irregularity that alters the direction of movement, and exhibits a flat velocity profile.

The term “ventilating” or “ventilation” is the process of exchanging or replacing air in any space to provide air circulation, temperature control, oxygen replenishment, and removal of moisture, odors, smoke, heat, dust, airborne infectious agents, various gases, and the like.

As used herein, the term “contaminate” or “contamination” is a constituent, impurity, or some other undesirable element that spoils, corrupts, infects, makes unfit, or makes inferior a material, physical body, or given environment. As used herein, the term “disease” refers to an abnormal condition that negatively affects the structure and/or function of an organism, which is not due to an acute external injury or trauma. The term “communicable disease” refers to an illness caused by an infectious agent or its toxins that occurs through direct or indirect transmission of the infectious agent or its products from an infected individual or via an animal, vector, or the inanimate environment to a susceptible animal or human host. The term “contagious” refers to a period during which an individual is able to transmit an infectious agent or its toxins at a load sufficient to infect another individual.

The term “lateral” refers to directions or positions relative to an object spanning the width of a body of the object, relating to the sides of the object, and/or moving in a sideways direction with respect to the object. The term “longitudinal” refers to directions or positions relative to an object spanning the length of a body of the object; relating to the top or bottom of the object, and/or moving in an upwards and/or downwards direction with respect to the object. The term “linear” refers to directions or positions relative to an object following a straight line with respect to the object, and/or refers to a movement or force that occurs in a straight line rather than in a curve. The term “lineal” refers to directions or positions relative to an object following along a given path with respect to the object, wherein the shape of the path is straight or not straight.

The terms “flexible,” “flexibility,” and/or “pliability” refer to the ability of an object or material to bend or deform in response to an applied force; “the term “flexible” is complementary to “stiffness.” The term “stiffness” and/or “rigidity” refers to the ability of an object to resist deformation in response to an applied force. The term “elasticity” refers to the ability of an object or material to resist a distorting influence or stress and to return to its original size and shape when the stress is removed. Elastic modulus (a measure of elasticity) is a property of a material, whereas flexibility or stiffness is a property of a structure or component of a structure and is dependent upon various physical dimensions that describe that structure or component.

The term “wear” refers to the phenomenon of the gradual removal, damaging, and/or displacement of material at solid surfaces due to mechanical processes (e.g., erosion) and/or chemical processes (e.g., corrosion). Wear causes functional surfaces to degrade, eventually leading to material failure or loss of functionality. The term “wear” as used herein may also include other processes such as fatigue (e.g., the weakening of a material caused by cyclic loading that results in progressive and localized structural damage and the growth of cracks) and creep (e.g., the tendency of a solid material to move slowly or deform permanently under the influence of persistent mechanical stresses). Mechanical wear may occur as a result of relative motion occurring between two contact surfaces. Wear that occurs in machinery components has the potential to cause degradation of the functional surface and ultimately loss of functionality. Various factors, such as the type of loading, type of motion, temperature, lubrication, and the like may affect the rate of wear.

The term “circuitry” refers to a circuit or system of multiple circuits configurable to perform a particular function in an electronic device. The circuit or system of circuits may be part of, or include one or more hardware components, such as a logic circuit, a processor (shared, dedicated, or group) and/or memory (shared, dedicated, or group), an Application Specific Integrated Circuit (ASIC), a field-programmable gate array (FPGA), programmable logic device (PLD), System-on-Chip (SoC), System-in-Package (SiP), Multi-Chip Package (MCP), digital signal processor (DSP), etc., that are configurable to provide the described functionality. In addition, the term “circuitry” may also refer to a combination of one or more hardware elements with the program code used to carry out the functionality of that program code. Some types of circuitry may execute one or more software or firmware programs to provide at least some of the described functionality. Such a combination of hardware elements and program code may be referred to as a particular type of circuitry.

As used herein, the term “element” may refer to a unit that is indivisible at a given level of abstraction and has a clearly defined boundary, wherein an element may be any type of entity. The term “entity” may refer to (1) a distinct component of an architecture or device, or (2) information transferred as a payload. As used herein, the term “device” may refer to a physical entity embedded inside, or attached to, another physical entity in its vicinity, with capabilities to convey digital information from or to that physical entity. The term “controller” may refer to an element or entity that has the capability to affect a physical entity, such as by changing its state or causing the physical entity to move.

The term “computer device” may describe any physical hardware device capable of sequentially and automatically carrying out a sequence of arithmetic or logical operations, equipped to record/store data on a machine readable medium, and transmit and receive data from one or more other devices in a communications network. A computer device may be considered synonymous to, and may hereafter be occasionally referred to, as a computer, computing platform, computing device, etc. The term “computer system” may include any type interconnected electronic devices, computer devices, or components thereof. Additionally, the term “computer system” and/or “system” may refer to various components of a computer that are communicatively coupled with one another. Furthermore, the term “computer system” and/or “system” may refer to multiple computer devices and/or multiple computing systems that are communicatively coupled with one another and configurable to share computing and/or networking resources. Examples of “computer devices,” “computer systems,” etc. may include cellular phones or smart phones, feature phones, tablet personal computers, wearable computing devices, an autonomous sensors, laptop computers, desktop personal computers, video game consoles, digital media players, handheld messaging devices, personal data assistants, an electronic book readers, augmented reality devices, server computer devices (e.g., stand-alone, rack-mounted, blade, etc.), cloud computing services/systems, network elements, in-vehicle infotainment (IVI), in-car entertainment (ICE) devices, an Instrument Cluster (IC), head-up display (HUD) devices, onboard diagnostic (OBD) devices, dashtop mobile equipment (DME), mobile data terminals (MDTs), Electronic Engine Management System (EEMS), electronic/engine control units (ECUs), electronic/engine control modules (ECMs), embedded systems, microcontrollers, control modules, engine management systems (EMS), networked or “smart” appliances, machine-type communications (MTC) devices, machine-to-machine (M2M), Internet of Things (IoT) devices, and/or any other like electronic devices. Moreover, the term “vehicle-embedded computer device” may refer to any computer device and/or computer system physically mounted on, built in, or otherwise embedded in a vehicle.

As used herein, the term “network element” may be considered synonymous to and/or referred to as a networked computer, networking hardware, network equipment, router, switch, hub, bridge, radio network controller, radio access network device, gateway, server, and/or any other like device. The term “network element” may describe a physical computing device of a wired or wireless communication network and be configurable to host a virtual machine. Furthermore, the term “network element” may describe equipment that provides radio baseband functions for data and/or voice connectivity between a network and one or more users. The term “network element” may be considered synonymous to and/or referred to as a “base station.” As used herein, the term “base station” may be considered synonymous to and/or referred to as a node B, an enhanced or evolved node B (eNB), next generation nodeB (gNB), base transceiver station (BTS), access point (AP), roadside unit (RSU), etc., and may describe equipment that provides the radio baseband functions for data and/or voice connectivity between a network and one or more users. As used herein, the terms “vehicle-to-vehicle” and “V2V” may refer to any communication involving a vehicle as a source or destination of a message. Additionally, the terms “vehicle-to-vehicle” and “V2V” as used herein may also encompass or be equivalent to vehicle-to-infrastructure (V2I) communications, vehicle-to-network (V2N) communications, vehicle-to-pedestrian (V2P) communications, or V2X communications.

As used herein, the term “channel” may refer to any transmission medium, either tangible or intangible, which is used to communicate data or a data stream. The term “channel” may be synonymous with and/or equivalent to “communications channel,” “data communications channel,” “transmission channel,” “data transmission channel,” “access channel,” “data access channel,” “link,” “data link,” “carrier,” “radiofrequency carrier,” and/or any other like term denoting a pathway or medium through which data is communicated. Additionally, the term “link” may refer to a connection between two devices through a Radio Access Technology (RAT) for the purpose of transmitting and receiving information.

The foregoing description of one or more implementations provides illustration and description of various example embodiment, but is not intended to be exhaustive or to limit the scope of embodiments to the precise form disclosed. Modifications and variations are possible in light of the above teachings or may be acquired from practice of various embodiments. Where specific details are set forth in order to describe example embodiments of the disclosure, it should be apparent to one skilled in the art that the disclosure can be practiced without, or with variation of, these specific details. The description is thus to be regarded as illustrative instead of limiting.

1. A protective air wall unit (PAWU) for use in an enclosed space, the PAWU comprising:
a pressure generator configured to generate an airflow;
a decontamination device coupled to the pressure generator; and
a control unit communicatively coupled with the pressure generator, the control unit operable to control the pressure generator to generate the airflow to flow through the enclosed space such that an air barrier is created that prevents a contaminate from crossing the air barrier and carries the contaminate to the decontamination device,
wherein the decontamination device is configured to reduce or prevent the contaminate from recirculating through the enclosed space.

2. The PAWU of claim 1, wherein the pressure generator comprises an airflow output and an airflow input, and the decontamination device coupled to the airflow input of the pressure generator, and wherein the airflow recirculates to the pressure generator via the airflow input after the decontamination device substantially removes the contaminate.

3. The PAWU of claim 2, wherein the PAWU is coupled to a first end of a first duct and a first end of a second duct, and the control unit is further operable to:
control the pressure generator to generate an airflow to flow through the first duct, out of a first nozzle coupled to a second end of the first duct, and through a second nozzle coupled to a second end of the second duct,
wherein the air barrier is created between the first nozzle and the second nozzle.

4. The PAWU of claim 3, further comprising:
a first sensor configured to measure a pressure level of the airflow output by the pressure generator; and
a second sensor configured to measure a quality or operation of the decontamination device.

5. The PAWU of claim 4, wherein the decontamination device comprises an air filter, wherein the air filter is one or more of a paper filter, polyurethane foam filter, cotton filter, stainless steel mesh filter, fiberglass filter, a high-efficiency particulate absorbing (HEPA) air filter, a semi-HEPA filter, an Ultra-Low Particulate Air (ULPA) filter, or a semi-ULPA filter.

6. The PAWU of claim 4, wherein the decontamination device comprises an ultraviolet germicidal irradiation (UVGI) system.

7. The PAWU of claim 4, wherein the decontamination device comprises an electrostatic precipitator (ESP) system.

8. The PAWU of claim 4, wherein the second sensor comprises one or more of a pressure sensor, a particle sensor, a microwave cavity sensor, a electrochemical nanosensor, or a plasmonic photothermal biosensor.

9. The PAWU of claim 4, wherein the control unit is further operable to:
obtain one or both of first sensor data from the first sensor and second sensor data from the second sensor; and
adjust operational parameters of the pressure generator based on the first sensor data or the second sensor data.

10. The PAWU of claim 1, further comprising a flow regulator configured to control the pressure of the airflow, wherein the flow regulator comprises:
a restricting element configured to maintain a set pressure; and
a measurement element configured to measure the pressure of the airflow for the restricting element.

11. The PAWU of claim 1, wherein the pressure generator comprises one or more of an air pump, an air compressor, a vacuum pump, a fan, or an air knife.

12. The PAWU of claim 1, wherein the enclosed space is a cabin of a vehicle.

13. A vehicle, comprising:
a ducting system; and
a protective air wall unit (PAWU) coupled with the ducting system, the PAWU including:
a pressure generator configured to generate an airflow,
a decontamination device coupled to the pressure generator; and
a control unit communicatively coupled with the pressure generator, the control unit operable to control the pressure generator to generate the airflow to flow through an enclosed space of the vehicle via the ducting system such that an air barrier is created in the enclosed space that prevents a contaminate from crossing the air barrier and carries the contaminate to the decontamination device,
wherein the decontamination device is configured to reduce or prevent the contaminate from recirculating through the enclosed space.

14. The vehicle of claim 13, wherein the pressure generator further comprises an airflow output and an airflow input, and the decontamination device coupled to the airflow input of the pressure generator, and wherein the airflow recirculates to the pressure generator via the airflow input after the decontamination device substantially removes the contaminate.

15. The vehicle of claim 14, wherein the ducting system comprises an air supply duct and a recirculation duct, the airflow output is coupled to a first end of the air supply duct, and the airflow input is coupled to a first end of a recirculation duct, and the control unit is further operable to:
control the pressure generator to generate an airflow to flow through the air supply duct, out of an output nozzle coupled to a second end of the air supply duct, and through an input nozzle coupled to a second end of the recirculation duct, wherein the air barrier is created between the input nozzle and the output nozzle.

16. The vehicle of claim 14, further comprising:
a first sensor configured to measure a pressure level of the airflow output by the pressure generator; and
a second sensor configured to measure a quality or operation of the decontamination device.

17. The vehicle of claim 16, wherein the control unit is further operable to:
obtain one or both of first sensor data from the first sensor and second sensor data from the second sensor; and
adjust operational parameters of the pressure generator based on the first sensor data or the second sensor data.

18. The vehicle of claim 16, wherein the decontamination device comprises one or more of an air filter, an ultraviolet germicidal irradiation (UVGI) system, and an electrostatic precipitator (ESP) system, and the pressure generator comprises one or more of an air pump, an air compressor, a vacuum pump, a fan, or an air knife.

19. The vehicle of claim 16, wherein the first sensor comprises a pressure sensor, and the second sensor comprises one or more of a pressure sensor, a particle sensor, a microwave cavity sensor, a electrochemical nanosensor, or a plasmonic photothermal biosensor.

20. The vehicle of claim 13, further comprising a flow regulator configured to control the pressure of the airflow, wherein the flow regulator comprises:
a restricting element configured to maintain a set pressure; and
a measurement element configured to measure the pressure of the airflow for the restricting element.

21. A controller for operating a protective air wall unit (PAWU) for use in an enclosed space, the PAWU comprising a pressure generator configured to generate an airflow and a decontamination device coupled to the pressure generator configured to reduce or prevent a contaminate from recirculating through the enclosed space, the control unit comprising:
processor circuitry configurable to determine operational parameters for generation of the airflow to flow through the enclosed space such that an air barrier is created that prevents the contaminate from crossing the air barrier and carries the contaminate to the decontamination device; and
interface circuitry configurable to send control signaling indicating the determined operational parameters, wherein the control signaling is to control the pressure generator to generate the airflow.

22. The controller of claim 21, wherein the PAWU is coupled to a first end of a first duct and a first end of a second duct, and the processor circuitry is further configurable to:
control the pressure generator to generate an airflow to flow through the first duct, out of a first nozzle coupled to a second end of the first duct, and through a second nozzle coupled to a second end of the second duct, wherein the air barrier is created between the first nozzle and the second nozzle.

23. The controller of claim 21, wherein the processor circuitry is further configurable to:
obtain first sensor data from a first sensor, the first sensor data representative of measured pressure levels of an airflow output by the pressure generator;
obtain second sensor data from a second sensor, the second sensor data representative of measured quality or operation of the decontamination device; and
adjust the operational parameters based on the first sensor data or the second sensor data.

24. The controller of claim 23, wherein the first sensor comprises a pressure sensor, and the second sensor comprises one or more of a pressure sensor, a particle sensor, a microwave cavity sensor, a electrochemical nanosensor, or a plasmonic photothermal biosensor.

25. The controller of claim 21, wherein the decontamination device comprises an ultraviolet germicidal irradiation (UVGI) system or an electrostatic precipitator (ESP) system, and wherein:
the processor circuitry is configurable to determine other operational parameters for controlling the UVGI or the ESP system; and
the interface circuitry is configurable to send other control signaling indicating the determined other operational parameters, the other control signaling to control the UVGI or the ESP system.
